(19) United States
(12) Patent Application Publication
(10) Pub. No.: US 2017/0357754 A1
(43) Pub. Date: Dec. 14, 2017

(54) CONTROL OBJECT FOR CONTROLLING A TRANSFER OF DUAL-ENERGY CT IMAGE DATA TO A CLIENT DEVICE

(71) Applicant: Siemens Healthcare GmbH, Erlangen (DE)

(72) Inventors: Peter HUBER, Windsbach (DE); Stefan THESEN, Dormitz (DE)

(73) Assignee: Siemens Healthcare GmbH, Erlangen (DE)

(21) Appl. No.: 15/615,902

(22) Filed: Jun. 7, 2017

(30) Foreign Application Priority Data
Jun. 10, 2016 (DE) 102016210312.1

(51) Int. Cl. G06F 19/00; G06Q 50/22; A61B 6/03; G06T 7/00; H04L 29/08

(52) U.S. Cl. CPC G06F 19/321; G06T 7/0012; H04L 67/06; H04L 67/32; A61B 6/03; G06Q 50/22

(57) ABSTRACT

A system and method for processing of dual-energy image data measurements are disclosed. The image data acquired on a computed tomograph is collected together to form a container before being sent to client devices for post-processing. The container contains a control object for the respective image dataset, which includes evaluation specifications and post-processing specifications for post-processing of the image data on the client device.

### PRIORITY STATEMENT

The present application hereby claims priority under 35 U.S.C. §119 to German patent application number DE 102016210312.1 filed Jun. 10, 2016, the entire contents of which are hereby incorporated herein by reference.

### FIELD

At least one embodiment of the present invention generally relates to the transfer of image datasets of dual-energy measurements to at least one client device and in particular relates to methods and systems for controlling the processing of medical imaging data, which can involve the presentation of vessels of the head for example.

### BACKGROUND

In medical imaging the standard that is predominantly used nowadays is the DICOM standard (Digital Imaging and Communications in Medicine—DICOM). It relates to the digital processing of images and communications in medicine and is an open standard for storage and for exchange of information in medical imaging data management. This information can be digital images, additional information such as segmentations, surface definitions or image registrations, for example.

DICOM standardizes both the format for storing the data and also the communications protocol for its exchange. On an abstract level this format is comparable with other image formats, such as JPEG or the like. The image data and its attributes and metadata (e.g. image width, bit depth), which are necessary for presentation or contain the information about the creation of the image (e.g. modality, scan mode etc.), are stored. The data/images themselves however do not contain any algorithms or program codes for presenting the data.

Within the framework of the development of the imaging systems, methods have been invented for which a suitable post-processing and/or visualization must be employed, in order to obtain the full diagnostic information from the image data. Examples of these techniques are perfusion measurements, dual-energy methods, etc. The image data is acquired on an image acquisition system (e.g. on a CT scanner) and is usually post-processed on one or more (other) client device(s). The image acquisition system and the client device exchange data with each other via a network interface.

Suitable methods for processing the image data are dependent on the technical parameters and conditions of the respective client device. If a user at their client device wishes to modify these post-processings and visualizations interactively during diagnosis, then this is disadvantageously not possible with the systems currently known at a generic diagnosis workstation. The user must then change the software used and/or the workstation and employ special proprietary programs that are frequently specific to the modality. To do this the user must also know which DICOM images/image data must be loaded into an application in which way. For a dual-energy evaluation for example the correct image series must be identified, transferred and loaded into the right application. In some cases further settings are also necessary (e.g. selection of an evaluation method). Once the evaluation on the client device is concluded, further result objects (e.g. in the form of static result images) are mostly created and set up in DICOM format.

Furthermore there are data objects such as CT or MR data, which cannot be displayed at all with standard viewers. This is an important disadvantage of known systems, since the post-processing and display is thus only possible on the client device under very restricted conditions.

### SUMMARY

At least one embodiment of the present invention is directed to a system, and at least one embodiment is directed to a method, with which the post-processing of medical image data can be improved and in particular designed in a more flexible manner. Preferably client devices are to be extended such that a post-processing of image data becomes possible, without there being a requirement for a specific technical device construction or device configuration on the client device (e.g. implementation of specific viewing software).

Embodiments of the are directed to a system, a processing unit, an image acquisition unit and two processing methods (for pre-processing and post-processing).

The embodiments are described below on the basis of the method. Features, advantages or alternate forms of embodiment mentioned here are likewise to be transferred to the other claimed subject matter and vice versa. In other words the physical claims (which are directed to a system or to a computer program or to a product for example) can also be further developed with the features that are described or claimed in conjunction with the method. The corresponding functional features of the method are embodied in such cases by corresponding physical modules, in particular by electronic hardware modules or microprocessor modules, of the system or vice versa.

At least one embodiment of the inventive system is designed for a plurality of different terminals (viewing devices, diagnosis stations, referred to as ‘client device’ below) and serves to extend the client devices such that a post-processing of different medical image data becomes possible thereon. To this end the transfer technology of the image data from an image acquisition system to the client device is changed. The image data to be transferred is extended in accordance with the invention by additional information. In particular a container with a control object is created and transferred, in which a post-processing functionality for the respective image data is specifically incorporated.

An example embodiment of the invention relates to a method for pre-processing of image data. The pre-processing is used for transmission to one or more client device(s). The method can be carried out on the image acquisition system. The result of the pre-processing can be stored—preferably on the image acquisition system or centrally. The method comprises:
Acquisition of image data on the image acquisition system;
Determination of control specifications for post-processing and evaluation of the image data and storage of the same in a control object;
Creation of a container, comprising the acquired image data with the assigned control object; and
Storage of the created container.

In accordance with a further example embodiment, the present invention relates to a method for interactive post-processing of medical image data on a client device, comprising:
Reading-in of a container with image data and with a control object via an interface; and
Releasing the control object from the container, in order to control the post-processing of the image data with the control object.

In accordance with another embodiment, the invention thus relates to a system for processing of medical image data, comprising:
an image data acquisition system, in particular a dual-energy computed tomography system, which serves to acquire image data;
a processing unit, which serves to create a container, which comprises the image data and a control object uniquely assigned to the image data;
at least one client device, which is intended to detect the container and extract the control object from said container, in order to control the processing of the image data with the control object; and
a network for the exchange of data between the image data acquisition system, the processing unit and the at least one client device.

Example embodiments of the invention include two different methods, which are either carried out on the image acquisition device (or the processing unit) or on the client device.

The described methods can be provided as a computer program stored on a non-transitory medium, which comprises commands that are intended to carry out the respective method when the program is executed on the computer.

### BRIEF DESCRIPTION OF THE DRAWINGS

In the more detailed description of the figures given below, example embodiments with their features and further advantages, which are not to be understood as restrictive, are discussed with reference to the drawing.

FIG. 1 shows, in a schematic overview diagram, an image acquisition system exchanging data with a client device.

FIG. 2 shows a flow diagram for a method for creating a container.

FIG. 3 shows a flow diagram for a method for post-processing of image data on a client device.

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

In accordance with one embodiment, the invention thus relates to a system for processing of medical image data, comprising:
an image data acquisition system, in particular a dual-energy computed tomography system, which serves to acquire image data;
a processing unit, which serves to create a container, which comprises the image data and a control object uniquely assigned to the image data;
at least one client device, which is intended to detect the container and extract the control object from said container, in order to control the processing of the image data with the control object; and
a network for the exchange of data between the image data acquisition system, the processing unit and the at least one client device.

The image data acquisition system, in an example embodiment of the invention, is a dual-energy computed tomography system. Embodiments of the invention can however likewise be applied to other image acquisition devices, such as normal x-ray devices, CT and/or MRT systems.

The processing unit can be implemented in software or hardware and embodied on the image data acquisition system. As an alternative it can also be connected as a separate unit to the other modules of the system via an interface.

The client device can be a computer-based system. Advantageously it no longer has to have specific post-processing functionality available to it, since said functionality is delivered so-to-speak with the image data. The client device can be a Personal Computer, a mobile terminal (laptop, cell phone), a network consisting of computer-based entities, a diagnostic station or a viewing station. The client device does not necessarily have to be a component of a client/server architecture, but can be based on any given network architecture.

The client device is an electronic system with an interface for receiving image data, a processing unit and a display unit for presentation of the image data in accordance with the specifications, which in accordance with the invention are coupled to the image data. The client device serves as a data sink and is supplied with data from a data source (e.g. an image acquisition system). It is entirely possible (and also normal) for the image scanner (the data source) to send the image data with the assigned control object to more than one data sink (client device). There can also be provision for the image data with the control object to be sent or forwarded from a first data sink to further receiver nodes (e.g. first and further diagnostic workstations, PACS and other target systems).

The container involves a data container. The container is a digital object, which along with the image datasets, contains an extension that serves to provide a post-processing and visualization functionality at the recipient of the container, the respective client device. This functionality is provided in the control object and can be created and designed specifically for the image datasets transmitted in each case. The control object can be appended to the image data or combined with the image data in some other way (e.g. in the form of attributes or shadow attributes in a DICOM dataset).

Thus, for example, a first container for dual-energy image data contains a first control object with a set of control functions designed for the dual-energy data and a second container for x-ray data contains a second control object with a set of control functions designed for the x-ray data.

In a further form of embodiment of the invention there can optionally be provision for the container to be created specifically for the receiving client device. In this way a first container can be designed for a complex viewing station, while a second container is designed for a simple PC, which does not have any specific installations and hardware configurations available.

In accordance with an example embodiment of the invention, the control object serves to control the visualization of the image data on the client device. The visualization can be interactive, so that in addition masks are created, which, for post-processing of the image data, can be displayed on the client device and can be operated by the user. Advantageously this makes it possible to send the image data to any given recipient, which e.g. only has a standard viewer available or which provides no viewing functionality or no specific viewing functionality.

In accordance with a further example embodiment of the invention, the processing unit is embodied as a cloud server. This enables a further flexibility to be achieved, since the image acquisition systems can be connected via an interface to the processing unit and do not have to provide such a processing unit directly. It is therefore advantageously not necessary for the existing image acquisition systems to have to be extended or modified.

In accordance with a further example embodiment of the invention, the control object comprises an evaluation specification for the image data transferred in the container.

This evaluation specification can be provided directly in the form of program code (HTML or Web assemblies). As an alternative or cumulatively (for parts or extracts of the evaluation specification), the evaluation specification can also be provided as a reference to a program code that is accessible via a network interface. This makes it possible to change the evaluation specification without any change to the container or to the processing unit having to be carried out.

In accordance with a further example embodiment of the invention, the control object includes a transformation command. This serves to transform a data representation in the image data into pixel values on the client device.

In accordance with a further example embodiment of the invention, the control object serves to create a result object on the client device. The result object can comprise final rendered images. The result object can be adapted to the respective technical system conditions of the client device and e.g. comprise parameter sets for controlling the image presentation. The parameter sets can preferably be adapted to the image data that is transferred in the container.

In accordance with a further example embodiment of the invention, the control object comprises an interaction module. The interaction module is embodied to create and to apply a specific user interface adapted to the image data and to the client device for interaction with the image data presented or to be presented on the client device. The interaction module thus serves to create screen masks on the client device. The created screen masks can be organized dedicated both to the technical configurations of the client device and to the image data transmitted in the container. This enables the transmission capacity to be efficiently utilized, in that only the commands relevant for the recipient (client device) and for the image data transferred in the container are transmitted for post-processing and visualization. The screen masks can comprise windows for input and output of data. They can also serve to display a masked presentation of image data, in that e.g. a mask can be created automatically that covers all bone structures of the image data. The user can then edit and improve or adapt these masks. It is e.g. also possible, with the aid of the masks, to have the bones removed from a 3D presentation, in order thereby to obtain an “unobstructed view” of the vessels.

Interactions of the user at the client device are however not always connected with masks. There are also entirely mask-free interactions. For example if, in a CT, the iodine content in a ROI (region of interest) is to be displayed or if in a perfusion computation the brain region is to be changed, which is used for normalization.

An example embodiment of the invention relates to a method for pre-processing of image data. The pre-processing is used for transmission to one or more client device(s). The method can be carried out on the image acquisition system. The result of the pre-processing can be stored—preferably on the image acquisition system or centrally. The method comprises:
Acquisition of image data on the image acquisition system;
Determination of control specifications for post-processing and evaluation of the image data and storage of the same in a control object;
Creation of a container, comprising the acquired image data with the assigned control object; and
Storage of the created container.

The method can also comprise sending the created container to selected or to specific recipients (client devices).

In an advantageous development of an example embodiment of the invention, the evaluation specification comprises an executable evaluation code or a link that references the executable evaluation code.

In accordance with a further example embodiment, the present invention relates to a method for interactive post-processing of medical image data on a client device, comprising:
Reading-in of a container with image data and with a control object via an interface; and
Releasing the control object from the container, in order to control the post-processing of the image data with the control object.

In an advantageous development of an example embodiment of the invention, the post-processing of the image data is adapted to the image data and to the technical configurations of the client device. The method is preferably carried out at run time with the loading of the image data.

Example embodiments of the invention include at least two different methods, each carried out on the image acquisition device (or the processing unit) or on the client device. The methods of embodiments comprise on the one hand sections that are carried out on the image acquisition system and on the other hand sections that are carried out on the client device. The system accordingly comprises sections (in the sense of functional modules) that are implemented on the image acquisition system and on the other hand sections that are implemented on the client device. In accordance with a further embodiment, the invention can also relate to a system that only comprises the image acquisition device-related section or only comprises the client device-related section.

The described methods can be provided as a computer program stored on a non-transitory medium, which comprises commands that are intended to carry out the respective method when the program is executed on the computer.

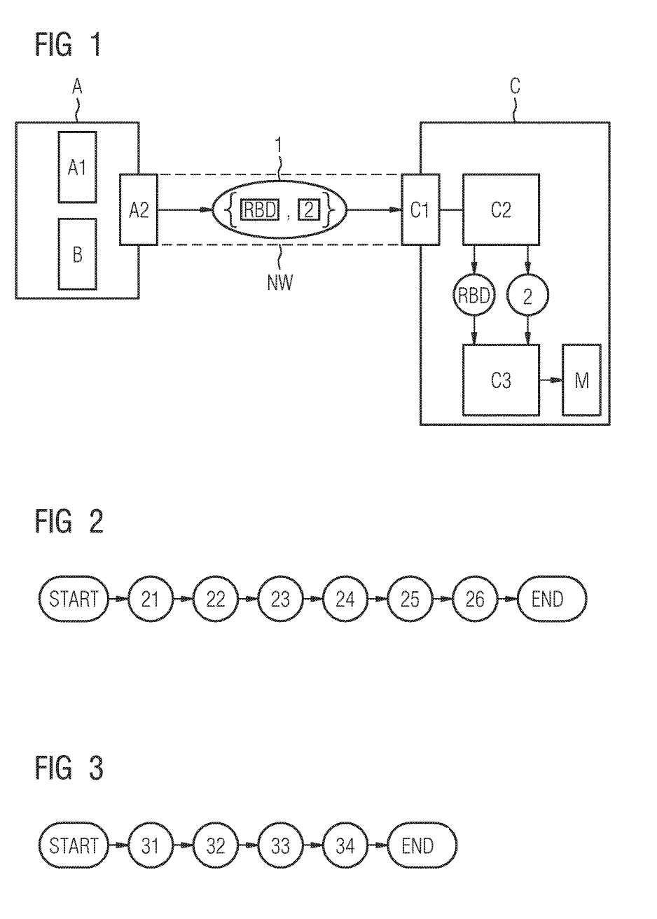

FIG. 1 shows a schematic overview diagram of an embodiment of the inventive system. The system comprises an image acquisition part with the image acquisition system A and a post-processing and visualization part, to which a client device C is assigned.

The image acquisition system A comprises an image data measurement unit A1 and serves to measure and to acquire image data RBD. In this case a dual-energy CT system can be involved for example. As an alternative the measurement data can also be read in from a memory and have already been acquired at an earlier point in time. A processing unit B serves to acquire evaluation specifications for post-processing for the acquired image data RBD, in order to create a control object 2 therefrom. The processing unit B can be integrated into the image acquisition system A (as is shown in the example embodiment depicted in FIG. 1). Preferably the processing unit B can also be connected as a separate entity via a network NW. The processing unit B can also be embodied as a Web server and, in this form of embodiment of the invention, is connected to the image acquisition system A and not implemented directly on the image acquisition device. This gives the advantage that neither the acquisition system A nor the client device C have to be changed, although the underlying processes, functions and computing specification are changed and can be dynamically changed.

The processing unit B serves to create a container 1. The container 1 comprises the image data RBD to be transmitted to a client device C in each case and a control object 2 dedicated and specifically assigned to this image data RBD. The created container 1 is transmitted via an output interface A2 and the network NW to a selected client device C.

An input interface C1 is located at the client device C, which is intended for reading-in the container 1 and for forwarding the same to an extractor C2. The extractor C2 is intended for extraction of the image data RBD and of the control object 2 from the container 1 and for forwarding the extracted data to a processor C3. The processor C3 is used for post-processing the received image data RBD, in that the control object 2 with an evaluation specification contained therein is applied to the image data RBD, in order to present the image data BD on a monitor M of the client device C.

In a preferred form of embodiment of the invention the evaluation specification can be adapted specifically to the respective image data RBD. In addition the evaluation specification can be adapted in a development of the invention cumulatively to the technical operating conditions of the client device C.

FIG. 2 describes an execution sequence for creation of the container 1. After the start of the method, in step 21, a type of the image datasets RBD acquired at the image acquisition system A is determined. In step 22 evaluation specifications for the type of image datasets RBD determined are acquired. In step 23 the technical parameters of the client system or client device C are acquired. In this case storage capacity, computer resources, post-processing functionality and further technical aspects of the client device C can be involved. In step 24 the evaluation specifications for the type of image datasets RBD acquired and for the acquired technical parameters are selected. This means that the selected evaluation specification is specifically designed both for the respective image datasets RBD (e.g. for the anatomical region, the type of data acquisition, the acquired section, the resolution etc.) and also for the technical operating conditions of the client device C (e.g. viewing functionality already available, memory, processor capacity etc.). In step 25 the container 1 is created with the image data RBD and the image-data-specific evaluation specification for this image data RBD. In step 26 the container 1 can be sent via the network NW to the client device C.

A method execution sequence for the post-processing of the image data RBD on the client device C, which refers to FIG. 3, is explained in greater detail below. After the start of the method, in step 31, the container 1 is received, in order to be extracted in step 32 in the extractor C2. In particular the image data RBD and the control object 2 that comprises an instruction object are extracted here. In step 33 the extracted control object with the evaluation specifications contained therein is applied to the extracted image datasets RBD, in order to create image datasets, which are able to be displayed in step 34 on a monitor of the client device C.

In general the aim of the present application is that image data RBD of medical devices (CT, MR, AX . . . ) is stored and transmitted with extensions (control object 2). The extension is used for post-processing, visualization and/or for evaluation of the image data RBD. The control object 2 can comprise an evaluation specification or can reference such a specification. This enables image data BD to be created, displayed and/or processed from the DICOM image data RBD, on the client device C. The image data is stored and transmitted jointly with the control object 2 with an extension (namely with an evaluation specification for evaluation of the image data). The extension comprises not only the image data (voxels) and attributes (matrix size, bit depth, date of recording etc.), but also the information (or a reference) for visualization and/or post-processing of the image data RBD. This produces from the image data RBD a container 1, which brings together data and method (function). This brings advantages in the handling. But it also allows standardized computers to be used as end customer systems, which do not have to be equipped specifically for particular post-processing functionalities. Furthermore standard technologies such as Web browsers can be used to bring specific functionalities (e.g. a new visualization mode or a dual-energy post-processing) to a standard workstation.

The user at the client device C no longer has to have any knowledge about the underlying data structures, about the post-processing software and about its operation if he wishes to post-process image data RBD. Thus for example it is no longer necessary to map image series on CT Cardiac in order to obtain a functional statement.

By contrast any given diagnostics or evaluation software for post-processing & visualization can be used, since the necessary algorithms & methods are contained in the container 1 or are referenced there. Advantageously a concrete version of a post-processing functionality for visualization/processing of the data can also be stored or referenced in the container 1. An update of a post-processing workstation/software no longer leads to changed results.

Data for which there is no standardized form of presentation (e.g. CT or MR data or input images) can be visualized and post-processed. The computing operations for post-processing and visualization can be relocated to a server or a cloud. In particular the reference contained in the data, as well as the algorithms or the reference to the algorithms, can likewise contain a reference to the server/the cloud.

The computing operations contained/referenced in the container 1 can be pre-computed on arrival at a computer system (or a pre-computation can be requested from a server/cloud). This means that the results are available to the user without any waiting time.

The cloud/server systems contained/referenced in the container 1 with the images could carry out the image transfer beforehand and in this way avoid waiting times. The use of clouds/servers can be undertaken with anonymized data, since the actual patient context is not necessary and can be created locally on the system of the client device C.

The method of at least one embodiment is especially suitable for new methods such as counting CT, in which data is created, the viewing of which in standardized DICOM viewers does not create any added value. So-called Image Call-Up methods can be undertaken simply with the aid of standard technology at generic workstations such as Web browsers. A skilled user can integrate their own processing and presentation functionalities via defined interfaces—without having to construct the complete diagnostic workstation infrastructure as such. In the method the compatibility with existing imaging standards can be maintained.

The system is explained below with reference to a dual-energy measurement for presentation of the vessels of the head. Typically nowadays two series with several hundred individual 2D DICOM images are stored. In accordance with at least one embodiment of the invention, the acquired image data RBD is stored in a DICOM multi-frame object, which contains the entire 2D data in one object. Furthermore other attributes are stored with the image. In one form of embodiment of the invention these attributes are proprietary shadow attributes. Preferably these attributes are also part of an extended standard.

Html/Javascript code is stored in these additional attributes of the control object 2, which allows the data to be rendered in a suitable manner. The code is executed by a standard Web browser, wherein the code as a data object transfers the DICOM image itself as reference. Furthermore the code also creates control elements within the framework of the interaction object. In the example given the html/Javascript code would compute from the data a specific calcium or bone mask specific for the image data RBD, which is presented on the monitor M for display and processes the corresponding input signals of the user. The corresponding data would then not be shown in the visualization. The visualization itself could e.g. be an average value image from the two series.

The control elements can for example allow the visualization of the bone mask as an overlay of a different color. Furthermore an interactive editing of the mask of the client device C is possible.

In a similar way the inclusive version of the html/Javascript code in the container 1 for the images could also be referenced. By calling up a page in a standard browser there could be the complete presentation incl. processing in a cloud. The cloud loads the DICOM data object itself in its turn directly from a clinic server. Standards, such as e.g. WADO for web-based DICOM access, can be used here. The patient context (e.g. name) can be displayed locally to the user.

In accordance with at least one embodiment of the invention specific masks, which can be edited, are generated for creation of new images.

Thus results can also be created on the client device C after the receipt of the container 1. Results can be final rendered images (in the sense of “secondary captures”). A result can also be a type of “presentation state”, which then consists of the required image data and a “frozen-in” parameter set for controlling the image presentation (image position, chosen overlay option, fusion blending value, LUT, activatable tools, etc.). Furthermore, during the course of the processing, auxiliary objects such as binary masks (editing results) and the like can be created. Such new objects can be made persistent and be used by users. The original data can optionally store references to such auxiliary objects and incorporate these if need be.

The created result images can then be transferred to the customer system in their turn by the embedded code; for example by a Web-based DICOM transfer in accordance with the WADO standard in the reverse direction.

User-specific settings can be managed with the system presented. In a concrete example this can be done with cookies for example, which are then stored in the browser or in the profile of the user.

Through the logical separation of image data and processing and presentation logic it is possible to combine the image data with a generic processing and presentation logic for a particular user group (e.g. “All Users”) and only to make possible more advanced premium presentation and processing variants for authenticated users (e.g. by login at a diagnosis station, cloud login, etc.). Thus a premium user, as well as the pure presentation of function overlays, could also carry out more advanced quantitative evaluation steps, if he has acquired the license previously offered.

The system can be linked into a generic portal solution (Web portal). The acquired and transmitted image data RBD itself “carries” its functionality intrinsically via the container 1.

By context-sharing methods it is possible also to realize more complex applications, e.g. synchronized scrolling covering various image segments, wherein the visualization functionality can be controlled from outside via the portal (image navigation, windowing, blending, presentation mode (MIP, VRT, MPR, etc.)).

Through the ad-hoc linking-in of visualization and processing options available online it is possible to detect usage patterns extending over a large user group and use them for future optimization decisions (usage tracking).

Furthermore the cloud-based processing of image data can make the image data available in an anonymized and abstract form. This can be used for the derivation of knowledge (learning-based algorithms, pattern recognition, similar cases, statistics).

Since the presentation methods and algorithms are themselves present in the data or are referenced, it is conceivable for non-validated or manipulated methods to be used, without the end user noticing this. This problem can be overcome by the information contained in the images for algorithm systems/visualization being signed with a PKI method. The presence of a valid signature can be displayed to the user.

The present application departs from the previous approach with a distribution of image data, parameters (DICOM) and algorithm systems (server/workstation). In accordance with the invention, with the container 1, the images intrinsically obtain an executable context for presentation and post-processing, which is no longer linked to a proprietary workstation.

The system preferably comprises an image acquisition section and a client section. The image acquisition section is assigned to image acquisition and relates to the modified creation of image data RBD to be transmitted and its storage. The client section is assigned to the post-processing of the image data on the client device C and serves to present the image data BD and to create a user interface with specific masks for the post-processing of the respective image data RBD.

Likewise within the framework of embodiments of the invention however, it only remains to provide the individual sections mentioned here, namely the image acquisition section for extension of the image acquisition system or the client section for extension of the client device system or end user system.

In accordance with of embodiments of the invention, not only is pre-implemented functional logic/business logic provided, as previously, on the client device C, but with the inventive system a defined extension interface for the client device C can be offered. This enables the program code transmitted additionally with the image file to extend or change the pre-installed program code. It is important here for this change to be able to be made at run time with the loading of the image data RBD. This part of the overall system is referred to below as the control system.

In this case the additional program code contains extensions, which allow the control system to present the images in various new ways. These presentations include the programmatic transformation of the data representation in the origin data (image data RBD) into pixel values on the presentation device. In such cases the data can be presented on a slice (2D), on a volume (3D) or also extending over points in time or modalities (4D). In concrete terms the data is transformed by a process, which loads the data into a memory of the computer and processes it on the basis of the program specification with the spatial and/or temporal environment.

Furthermore the program code can contain extensions, which allow the control system to create new results from the data. To this end the transferred data is loaded, transformed with the aid of the extension and then stored again or presented for display. Results can be a further image, structured information or masks. In concrete terms the transformation is carried out by a process that loads the data into the memory of the computer and computes it on the basis of the program specification with the spatial and/or temporal environment. Furthermore information from reference databases can be included in the computation. This can be of an anatomical nature for example (e.g. probability that after mapping onto a structure in standard coordinates (e.g. standard anatomy, especially head) there will be a bone present at this point).

Since the data intrinsically contains the extensions of the control system, the user also does not have to manually select any modes on the client device C, but is offered the extensions automatically. Likewise the facilities of the device as a whole can be extended via the dataset; i.e. after the installation.

At its heart the invention proposes a change in data storage, data processing and/or data transmission between an image acquisition system and one or more client device(s). The acquired image data RBD is no longer transferred directly and without an additional control object, in the way that it was acquired. In accordance with the invention the image data RBD is supplied to a processing unit B, which creates a container 1 uniquely for this input data or image data RBD, which comprises a control object as an extension.

In conclusion it should be pointed out that the description of the example embodiments of the invention and the example embodiments are basically not to be understood as restrictive in respect of a particular physical realization of the invention. All features explained and shown in conjunction with individual forms of embodiment of the invention can be provided in a different combination in the inventive subject matter, in order at the same time to realize their advantageous effects.

The area of protection of the present invention is given by the claims and is not restricted by the features explained in the description or shown in the figures.

For a person skilled in the art it is in particular evident that example embodiments of the invention cannot only be employed for dual-energy CT image acquisition devices but also for other medical image acquisition devices that require a specific post-processing or visualization of the image data. Furthermore the components or modules of the image acquisition system can also be realized distributed between a number of physical products, so that for example the processing unit can also be implemented at a central server.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. §112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A system for processing of medical image data, comprising:
an image data acquisition system, to acquire the medical image data;
a processing unit, to create a container, the container including the medical image data and an assigned control object;
at least one client device, to detect the container and to extract the assigned control object from the container, to control post-processing of the medical image data with the assigned control object; and
a network for exchange of data between the medical image data acquisition system, the processing unit and the at least one client device.

2. The system of claim 1, wherein the assigned control object serves to control a visualization of the medical image data on at least one of the at least one client device.

3. The system of claim 1, wherein the processing unit is embodied as a cloud server.

4. The system of claim 1, wherein the assigned control object includes an evaluation specification.

5. The system of claim 4, wherein the evaluation specification includes program code or a reference to the program code, accessible via a network interface.

6. The system of claim 1, wherein the assigned control object includes a transformation command, to transform a data representation in the medical image data into pixel values on the at least one client device.

7. The system of claim 1, wherein the assigned control object serves to create a result object on the at least one client device.

8. The system of claim 1, wherein the assigned control object includes an interaction module, embodied to create and to apply a specific user interface adapted to the medical image data and to the at least one client device for interaction with the medical image data presented on the at least one client device.

9. A processing unit, for use in a system for processing medical image data including an image data acquisition system to acquire medical image data, at least one client device and a network for exchange of data between the image data acquisition system, the processing unit and the at least one client device, the processing unit being configured to create a container, including the medical image data and an assigned control object.

10. An image acquisition system, comprising the processing unit of claim 9.

11. A method for pre-processing of medical image data, the method comprising:
acquiring the medical image data on an image acquisition system;
determining control specifications for processing and evaluating the medical image data, and storing of the medical image data in a control object;
creating a container, including the acquired medical image data and the control object, assigned to the acquired medical image data; and
storing the created container to complete the pre-processing of the acquired medical image data.

12. The method for pre-processing of claim 11, wherein the control specifications include an executable evaluation program code or a link that references the executable evaluation program code.

13. A method for post-processing of medical image data on a client device, comprising:
reading-in a container, with medical image data and a control object uniquely assigned to the medical image data, via an interface; and
releasing the control object from the container, to control the post-processing of the medical image data with the control object.

14. The method of claim 13, wherein the post-processing of the medical image data is adapted to the medical image data and to the client device and is carried out at run time with a loading of the medical image data.

15. The system of claim 2, wherein the processing unit is embodied as a cloud server.

16. The system of claim 2, wherein the assigned control object includes an evaluation specification.

17. The system of claim 16, wherein the evaluation specification includes program code or a reference to the program code, accessible via a network interface.

18. The system of claim 2, wherein the assigned control object includes a transformation command, to transform a data representation in the medical image data into pixel values on the at least one client device.

19. The system of claim 5, wherein the assigned control object includes a transformation command, to transform a data representation in the medical image data into pixel values on the at least one client device.

20. A system for pre-processing of medical image data, the system comprising:
an image acquisition system to acquire the medical image data;
a processor, to create a container including the acquired medical image data and an assigned control object and to determine control specifications for processing and evaluating the medical image data; and
a memory to store the created container to complete the pre-processing of the acquired medical image data.

21. The system of claim 20, wherein the control specifications include an executable evaluation program code or a link that references the executable evaluation program code.

22. A system for post-processing of medical image data on a client device, comprising:
an interface to read-in a container, including medical image data and a control object uniquely assigned to the medical image data; and
a processor to release the control object from the container, to control the post-processing of the medical image data with the control object.

23. The system of claim 22, wherein the post-processing of the medical image data is adapted to the medical image data and to the client device and is carried out at run time with a loading of the medical image data.
